(19) United States
(12) Patent Application Publication
(10) Pub. No.: US 2006/0046974 A1
(43) Pub. Date: Mar. 2, 2006

(54) N-acyl modified polysaccharides and pharmaceutical compositions comprising same

(76) Inventor: John Jason Gentry Mullins, Pleasanton, CA (US)

Correspondence Address: John J G Mullins, 1618 East Gate Way # 304, Pleasanton, CA, 94566, US

(21) Appl. No.: 11/214,332

(22) Filed: Aug. 28, 2005

Related U.S. Application Data
(60) Provisional application No. 60/605,935, filed on Aug. 30, 2004.
(60) Provisional application No. 60/605,931, filed on Aug. 30, 2004.

(51) Int. Cl. A61K 31/737

(52) U.S. Cl. 514/54

(57) ABSTRACT

This invention relates to novel N-acyl modified polysaccharides and pharmaceutical compositions comprising the modified polysaccharides. Further the invention relates to processes for using such compounds to inhibit lipases, to lower cholesterol, to lower the absorption of dietary fat, or as a dietary fiber supplement. In a preferred aspect of the invention the modified polysaccharides are capable of absorbing substantial amounts of oils or fats while dissipating in an aqueous solution. The invention also relates to a non-absorbable and essentially non-digestible polysaccharide fiber composition.

This application hereby claims the priority date filing dates from prior provisional application No. 60/605,935, filed Aug. 30, 2004, and prior provisional application No. 60/605,931, filed Aug. 30, 2004, each being prior applications by the same inventor.

### FIELD OF THE INVENTION

This invention relates to novel N-acyl modified polysaccharides and pharmaceutical compositions comprising the modified polysaccharides. Further the invention relates to processes for using such compounds to inhibit lipases, to lower cholesterol, to lower the absorption of dietary fat, or as a dietary fiber supplement. In a preferred aspect of the invention the modified polysaccharides are capable of absorbing substantial amounts of oils or fats while dissipating in an aqueous solution. The invention also relates to a non-absorbable and essentially non-digestible polysaccharide fiber composition.

### BACKGROUND OF THE INVENTION

The use of natural polysaccharide fibers as digestive supplements is well known, however, there is a need for improved fibers with fewer side effects. Physicians and other medical researchers have suggested that everyone lower their effective calories from fat to less than 20% of their caloric intake. In view of the popular low-carbohydrate diet trend, this has become even more of a problem. The issue is whether losing weight to lower one's risk for a heart attack and other complications can justify the risks of a high-fat diet with respect to heart disease. Accordingly, there is the need for a bio-friendly dietary supplement that will lower the absorption of fat, when a person consumes a diet fairly rich in fat.

More particularly, there is a need for non-absorbable and essentially non-digestible fibers having the ability to absorb fat or oils in the digestive system in an efficient manner without clumping of the fiber with other fibers having oil or fats absorbed thereon. Such clumping can cause compaction and constipation, or produce oily blobs that are not evenly distributed in digestive waste. Such clumping or oily blobs can also have the undesirable side effect of trapping significant amounts of oil absorbable vitamins, and require vitamin supplementation in patients consuming the fiber.

With the increasing interest in the treatment of high cholesterol, high triglycerides and obesity, and the popularity of the low-carbohydrate diet (sometimes a high-fat diet) there is a need for digestive fibers having improved properties, but this has proved illusive and difficult to obtain. Compounds that absorb oil tend to clump together and avoid water, making it difficult to hydrate the fiber. For example, Geltex patents (for example, U.S. Pat. Nos. 6,703,369, 6,572,850 and 6,562,329) relate to synthetic polymers that absorb oil, which can be useful as stool softeners to help with constipation. However, such compounds still tend to cause clumping of oil absorbing fiber and the absorbed oil can traps significant amounts of oil-soluble vitamins. The blobs can lead to blob-like areas occurring in sections of the human waste that lead to uncomfortable oily stool or anal discharge of blobs. Also, such compounds can also cause constipation if the low-hydration, absorbed oil and polymer are not evenly distributed in the digestive waste of the person who consumed the oil-absorbing polymer composition. Merely having hydrophilic and hydrophobic areas (block copolymer) on the polymer still leaves substantially hydrophobic areas open. These open areas can associate with corresponding hydrophobic areas on another copolymer chain. Such associations can lead to undesired clumping and reduced hydration of the digestive waste.

Another undesirable feature of these synthetic polymers is that they have no known biological source (such as a bacterium) that could digest the polymer if substantial amounts were consumed by persons and human waste containing them would need to disposed of in sewage. Since such polymers are not derived from natural substances, they are not really bio-friendly to the environment and may be difficult to properly dispose of in sewage.

According, there is a need for oil absorbing bio-friendly compositions that distribute evenly throughout the digestive system, particularly in digestive waste, and hydrate well.

In addition to obesity and undesired weight gain, high cholesterol has become a concurrent problem in overweight patients (or those on a high fat diet). Many of the pharmaceutical compositions currently used to lower cholesterol in patients have substantial side effects that cause physicians to hesitate before prescribing such medications to patients having moderate to almost high cholesterol. Without such side effects, more patients could have a proscriptive and preventative benefit by lowering cholesterol to a more moderate or lower level. Often patients already have a high risk of a heart attack, have had a heart attack or early warning signs of a heart attach, or have even had a medical intervention to prevent an attack (such as an arterial stent, angioplasty, or bypass surgery).

Systemic drugs that block the formation of cholesterol (such as statins) can have substantial undesirable side effects. In fact, some have been withdrawn from the market in recent years due to highly undesirable side effects. There are some less stringent and indirect ways of lowering cholesterol by removing dietary bile acids from the digestive system that would ordinarily be recycled by the body, but they can also have uncomfortable side effects. Such drugs bind bile salts until excreted from the body and require the body to withdraw cholesterol from the blood stream to produce more dietary bile acids. This produces a cholesterol-lowering effect. The typical quaternary ammonium synthetic polymer bile binding compounds that indirectly reduce cholesterol by removing digestive bile often cause uncomfortable bloating, constipation and other undesired side effects. Therefore, in view of such side effects, physicians also hesitate before prescribing them unless a patient already has a cholesterol level that is quite high.

Some people, who do not have cholesterol high enough to warrant treatment with medications that have substantial side effects, have turned to Chitosan as a dietary fiber. Chitosan is a modified natural polysaccharide fiber, but results with this fiber have proved not highly effective. Chitosan typically only absorbs about 3-4% of its weight in dietary fat and does not dissipate evenly in the digestive fluids and waste. It has the tendency to form oily blobs that trap oil-absorbable vitamins and can require one to take a vitamin supplement. Even so, the annual world-wide market for Chitosan as a dietary fiber is in the hundreds of millions of dollars in recent years.

Accordingly, there is a need in the art for a bio-friendly dietary supplement that will lower cholesterol while simultaneously reducing the amount of absorbed calories from fat. Preferably, the dietary supplement will be more effective than Chitosan, or minimize its side effects.

There is a need in the art for improved oil absorbing compositions that distribute evenly in digestive waste and hydrate well, as well as improved anti-adiposity compositions which do not require an absolute low-fat diet in order to lower the absorption of dietary fat as calories, and anti-cholesterol compositions and methods. Preferably, such fibers will have a a molecular weight greater than 8 kDa and is not absorbed and not digested by a mammal.

### SUMMARY OF THE INVENTION

In one aspect the present invention provides a pharmaceutical composition comprising a polysaccharide dietary fiber that can absorb more than 8 times is weight in dietary fat in oil in an amount effective to lower serum cholesterol, and a pharmaceutically acceptable carrier. In a preferred embodiment the polysaccharide fiber is a poly-D-glucosamine derivative, wherein at least one hydrogen atom on from 3% to 10% on the amine groups (—NH2 groups) of the repeating D-glucosamine or modified D-glucosamine groups have been replaced by a 4-20 carbon atom alkyl group comprising at least one carboxylic group (preferably a terminal carboxylic group), wherein the carboxylic group may be esterfied by a lower alcohol group or may be in a salt form, in order to form N-alkylacyl groups, or modified N-alkylacyl groups, on the polymer backbone. Even more preferably, the N-alkylacyl group on the modified poly-D-glucosamine backbone is an N-6-hexanoic acid group, an N-8-octanoic acid group, an N-11-undecanoic group, or a combination thereof, wherein the acid group many be the free acid, an ester, or a salt thereof.

In another aspect the present invention provides a higher molecular weight Chitosan that is still water soluble or easily water dispersible in the digestive fluids and digestive waste with the ability to bind large amounts of dietary fat. Preferably the Chitosan has a viscosity greater than 50 cps, which is much greater than typical water soluble Chitosan products. Preferably, longer chains are provided with more surface area and compatibility for binding longer chain fatty acids that is not as possible with shorter chain Chitosan products.

In another aspect the present invention provides a process for lowering the serum cholesterol in a patient by treating the patient with an effective amount of the above composition.

In still another aspect, the present invention provides a method of increasing the digestive health of a mammal by treating said mammal with an amount of the above composition that is effective as a dietary fiber. Preferably, the composition is administered to the mammal in a dosage from about 500 milligrams to 3 grams per meal, preferably 750 milligrams to 2 grams per meal, and more preferably from 750 mg to 1 g per meal.

In another aspect the present invention relates to a pharmaceutical composition as described above, further comprising an amount of a lipase inhibitor effective for the treatment of obesity in admixture therewith.

### DETAILED DESCRIPTION OF THE INVENTION

Definitions

In accordance with the present invention and as used herein, the following terms are defined with the following meanings, unless explicitly stated otherwise.

The term “alkenyl” refers to a trivalent straight chain or branched chain unsaturated aliphatic radical. The term “alkinyl” (or “alkynyl”) refers to a straight or branched chain aliphatic radical that includes at least two carbons joined by a triple bond. If no number of carbons is specified alkenyl and alkinyl each refer to radicals having from 2-20 carbon atoms.

The term “alkyl” refers to saturated aliphatic groups including straight-chain, branched-chain and cyclic groups having the number of carbon atoms specified, or if no number is specified, having up to 20 carbon atoms. The term modified alkyl group means that one or more hydrogen atoms on the alkyl chain have been substituted with a lower alkyl, an alcohol group, an amino group, a halo group, a cycloalkyl, an aryl, or some other substituent that does not substantially interfere with the desired fat-absorbing and water dispersing traits of the of the overall molecule. In the modified alkyl group, 2 or 4 hydrogen atoms can be replaced by a double or triple bond, respectively. The term “cycloalkyl” as used herein refers to a mono-, bi-, or tricyclic aliphatic ring having 3 to 14 carbon atoms and preferably 3 to 7 carbon atoms.

As used herein, the terms “carbocyclic ring structure” and “C₃₋₁₆ carbocyclic mono, bicyclic or tricyclic ring structure” or the like are each intended to mean stable ring structures having only carbon atoms as ring atoms wherein the ring structure is a substituted or unsubstituted member selected from the group consisting of: a stable monocyclic ring which is aromatic ring (“aryl”) having six ring atoms; a stable monocyclic non-aromatic ring having from 3 to 7 ring atoms in the ring; a stable bicyclic ring structure having a total of from 7 to 12 ring atoms in the two rings wherein the bicyclic ring structure is selected from the group consisting of ring structures in which both of the rings are aromatic, ring structures in which one of the rings is aromatic and ring structures in which both of the rings are non-aromatic; and a stable tricyclic ring structure having a total of from 10 to 16 atoms in the three rings wherein the tricyclic ring structure is selected from the group consisting of: ring structures in which three of the rings are aromatic, ring structures in which two of the rings are aromatic and ring structures in which three of the rings are non-aromatic. In each case, the non-aromatic rings when present in the monocyclic, bicyclic or tricyclic ring structure may independently be saturated, partially saturated or fully saturated. Examples of such carbocyclic ring structures include, but are not limited to, cyclopropyl, cyclobutyl, cyclopentyl, cyclohexyl, adamantyl, cyclooctyl, [3.3.0]bicyclooctane, [4.3.0]bicyclononane, [4.4.0]bicyclodecane (decalin), 2.2.2]bicyclooctane, fluorenyl, phenyl, naphthyl, indanyl, adamantyl, or tetrahydronaphthyl (tetralin). Moreover, the ring structures described herein may be attached to one or more indicated pendant groups via any carbon atom which results in a stable structure. The term “substituted” as used in conjunction with carbocyclic ring structures means that hydrogen atoms attached to the ring carbon atoms of ring structures described herein may be substituted by one or more of the substituents indicated for that structure if such substitution(s) would result in a stable compound.

The term “aryl” which is included with the term “carbocyclic ring structure” refers to an unsubstituted or substituted aromatic ring, substituted with one, two or three substituents selected from loweralkoxy, loweralkyl, loweralkylamino, hydroxy, halogen, cyano, hydroxyl, mercapto, nitro, thioalkoxy, carboxaldehyde, carboxyl, carboalkoxy and carboxamide, including but not limited to carbocyclic aryl, heterocyclic aryl, and biaryl groups and the like, all of which may be optionally substituted. Preferred aryl groups include phenyl, halophenyl, loweralkylphenyl, napthyl, biphenyl, phenanthrenyl and naphthacenyl.

The term “arylalkyl” which is included with the term “carbocyclic aryl” refers to one, two, or three aryl groups having the number of carbon atoms designated, appended to an alkyl group having the number of carbon atoms designated. Suitable arylalkyl groups include, but are not limited to, benzyl, picolyl, naphthylmethyl, phenethyl, benzyhydryl, trityl, and the like, all of which may be optionally substituted.

The terms “halo” or “halogen” as used herein refer to Cl, Br, F or I substituents. The term “haloalkyl”, and the like, refer to an aliphatic carbon radicals having at least one hydrogen atom replaced by a Cl, Br, F or I atom, including mixtures of different halo atoms. Trihaloalkyl includes trifluoromethyl and the like as preferred radicals, for example.

The term “methylene” refers to —CH₂—.

The term “pharmaceutically acceptable salts” includes salts of compounds derived from the combination of a compound and an organic or inorganic acid. These compounds are useful in both free base and salt form. In practice, the use of the salt form amounts to use of the base form; both acid and base addition salts are within the scope of the present invention.

“Pharmaceutically acceptable acid addition salt” refers to salts retaining the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic nontoxic bases are isopropylamine, diethylamine, ethanolamine, trimethamine, dicyclohexylamine, choline, and caffeine.

“Biological property” for the purposes herein means an in vivo effector or antigenic function or activity that is directly or indirectly performed by a compound of this invention that are often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

In the compounds of this invention, carbon atoms bonded to four non-identical substituents are asymmetric. Accordingly, the compounds may exist as diastereoisomers, enantiomers or mixtures thereof. The syntheses described herein may employ racemates, enantiomers or diastereomers as starting materials or intermediates. Diastereomeric products resulting from such syntheses may be separated by chromatographic or crystallization methods, or by other methods known in the art. Likewise, enantiomeric product mixtures may be separated using the same techniques or by other methods known in the art. Each of the asymmetric carbon atoms, when present in the compounds of this invention, may be in one of two configurations (R or S) and both are within the scope of the present invention.

### Preferred Embodiments

In one aspect the present invention relates to novel pharmaceutical compositions comprising an effective amount of the modified poly-D-glucosamine non-absorbable fiber derivatives, wherein the fiber is capable of absorbing more than 10 times its weight in dietary fat or oil and is effective in lowering serum cholesterol when consumed with meals where the dietary fat content exceeds 20% of the caloric intake at that meal. In one embodiment, from 3% to 15%, preferably, 3% to 10% and more preferably about 5% to 8% of the amino groups on the poly-D-glucosamine (or on the modified poly-D-glucosamine) are replaced with a substituent moiety having a terminal acyl group. Preferably the substituent moiety that modifies the poly-D-glucosamine backbone is either an N-alklyacyl moiety or a modified N-alkylacyl moiety, wherein the alkyl portion contains at least 3 carbon atoms. The alkyl moieties may be the same or different and can be modified alkyl groups. The term modified alkyl group means that one or more hydrogen atoms on the alkyl chain have been substituted with a lower alkyl, an alcohol group, an amino group, a halo group, a cycloalkyl, an aryl, or some other compatible substituent that does not substantially interfere with the desired fat-absorbing and water dispersing traits of the of the overall molecule. In the modified alkyl group, 2 or 4 hydrogen atoms can also be replaced by a double or triple bond, respectively, to yield an alkenyl or alkynyl moiety. The term “cycloalkyl” as used herein refers to a mono-, bi-, or tricyclic aliphatic ring having 3 to 14 carbon atoms and preferably 3 to 7 carbon atoms. The pharmaceutical compositions may also comprise other therapeutic components such as a lipase inhibitor, a cortisol hormone inhibitor, a carbohydrate blocking component, a stimulant, vitamins and the like. Moreover, the pharmaceutical composition may be in combination with a pharmaceutically acceptable carrier or diluent, and may further comprise an effective amount of a lipophilic, non-absorbable biocompatible, pharmaceutically acceptable oil absorbing polymer. The additional therapeutic components may be present in the optimum dosages that are well known in the art, or readily determined by a skilled practitioner in this field. Other pharmaceutically acceptable fibers or fillers may be present.

In another aspect, the pharmaceutical compositions may be formulated into foodstuffs to provide a sports supplement beverage or solid foodstuff. The effective amount for such compositions can be readily determined based upon the information provided herein by routine experimentation or by the directions provided herein.

In another aspect the present invention provides a process for lowering the serum cholesterol in a patient by treating the patient with an effective amount of the above composition. The effective amount can be readily determined by a number of in vitro and in vivo tests as compared to the current anti-cholesterol medications that are approved by the FDA. It is not critical that the effectiveness be the same as commercial therapeutics for supplements or as medicaments for persons that would have a cholesterol level that is too low to ordinarily justify the side effects of commercially available therapeutics. Even a person who has a normal cholesterol level, a moderately elevated or a high normal cholesterol level could be provided with healthy benefit from the moderate cholesterol lowering effects of the present compositions.

In still another aspect, the present invention provides a method of increasing the digestive health of a mammal by treating said mammal with an amount of the above composition that is effective as a dietary fiber. Preferably, the composition is administered to the mammal in a dosage from about 500 milligrams to 3 grams per meal, preferably 750 milligrams to 2 grams per meal, and more preferably from 750 mg to 1 g per meal. Many persons have the need to increase their dietary fiber intake in a way that would either promote regularity, provide healthy bowel digestive activity, or both. The present compositions provide a naturally modified fiber supplement that is bio-friendly and is broken down by bacteria in the bowel and in waste disposal systems. By have both oil friendly and water friendly components in the fiber, this promotes a more even distribution of fiber throughout the waste which can minimize constipation side effects that some fiber can have.

In another aspect the present invention relates to a pharmaceutical composition as described above, further comprising an amount of a lipase inhibitor effective for the treatment of obesity in admixture therewith. As mentioned above, a number of therapeutics can be added to the present cholesterol lowering and fiber supplement compositions. One particularly preferred embodiment includes a lipase inhibitor for a synergistic effect in combination with the present fiber composition. More preferred is such a fiber composition that includes a lipase inhibitor and a carbohydrate metabolism blocker in amounts effective to treat obesity.

In a preferred embodiment, the fiber is modified to absorb both oil and water and disperse in digestive materials instead of forming a gel, oily blob, or other gel-like composition. By evenly dispersing in the aqueous environment, the fiber promotes a more even distribution of itself in the digestive waste and minimizes any trapping of oil soluble vitamins within the gel, oily blob or other gel-like composition.

Preparation of Compounds

The poly-D-glucosamine or modified poly-D-glucosamine, or other compatible polysaccharide fibers, which are non-absorbable are readily available to one in the art. The alkylacyl groups for modifying the amino groups on the D-glucosamine moieties are readily available or can be synthesized using routine skill. Halo substituted alkylacyl groups can be synthesized or obtained. For example, 6-hexanoic acid, 8-octanoic and 11-undecanoic acid are all available from commercial vendors, such as Aldrich Chemical Company. Amination reactions to form secondary and or tertiary amines by coupling are well known in the art. Compound purification methods are described and referenced in standard textbooks

Starting materials used in any of these methods are commercially available from chemical vendors such as Aldrich, Sigma, Nova Biochemicals, Bachem Biosciences, and the like, or may be readily synthesized by known procedures.

Reactions are carried out in standard laboratory glassware and reaction vessels under reaction conditions of standard temperature and pressure, except where otherwise indicated.

During the synthesis of these compounds, the functional groups may be protected by blocking groups to prevent cross reaction during the coupling procedure. Examples of suitable blocking groups and their use are described in “The Peptides: Analysis, Synthesis, Biology”, Academic Press, Vol. 3 (Gross, et al., Eds., 1981) and Vol. 9 (1987), the disclosures of which are incorporated herein by reference.

Lipase inhibitor moieties having a free hydroxy group such as tetrahydro-esterastin (3,5-hydroxy-2-hexadeca-7,10-dienoic 1,3-lactone), 3,5-dihydroxy-2-hexylhexadeca-7,10-dienoic 1,3-lactone, 3,5-di-hydroxy-2-hexylhexadecanoic 1,3-lactone, and the like, are easily coupled to a polymer moiety having free hydroxy groups such as cellulose, chitosan and other polysaccharides having free hydroxyl groups, or any known stand-alone lipase inhibitor may be used. Carbohydrate blocking compounds, such as white kidney bean extract and the like are well-known. Cortisol inhibitor compounds are also well known.

In one preferred aspect of the invention, the amino or alcohol groups of the polysaccharide moiety, such as chitosan, is reacted with one or more types of n-haloalkanoic acid (or an acyl ester derivative) such as n-bromohexanoic acid, n-chlorolauric acid, n-bromoundecanoic acid in a molar ratio 1:1 to 1:115, or the like, sufficient to attach an organic acyl side chain to 1 to 15%, preferably from 3 to 10% and more preferably about 5%, of the free alcohol groups, amino groups or alcohol and amino groups on the polysaccharide chain to provide an organic acyl group modified polysaccharide, such as an organic acyl group modified chitosan derivative. Preferably, a secondary amination reaction is utilized to covert a desired percentage of primary amines on the poly-D-glucosamine chain into secondary amines. In such case, the n-haloalkanoic acid, or other halo derivative organic acyl group, etherizes free hydroxyl groups, replaces a hydrogen atom on an amino group, or forms a ketone with an acid group on a previously modified polysaccharide compound to provide a modified poly-D-glucosamine compound that absorbs more than 8 times its weight in dietary fat or oil and disperses in an aqueous environment to promote even distribution of the fiber in dietary waste. Particularly preferred polymer moieties to be modified are polysaccharides having multiple amino groups for coupling, such as chitosan or a chitosan that has optionally been sulfonated to render the polysaccharide a lipase inhibitor compound. Etherification, amination and ketone formation procedures are well-known in the art and well within the routine skill of the ordinary practitioner. Further, other acyl moieties and the techniques for binding them to a poly-D-glucosamine polysaccharide fiber or the like, are well-known in the art. The preferred compounds also include their pharmaceutically acceptable isomers, hydrates, solvates, salts and prodrug derivatives.

Examples of haloacyl groups for modification of the primary amine groups include chloromethylbenzoic acid or an ester thereof, 3-bromopropanoic acid, 2-chloroacetic acid, 6-bromohexanoic acid or an ester thereof, 8-chlorooctanoic acid, 11-bromoundecanoic acid, 12-bromododecanoic acid or an ester thereof, other n-haloalkanoic acids or esters thereof, and the like.

Preferred amino modifying groups are bridging groups terminated with at least one bromo or chlorine group and the other terminus is an acyl group or an acyl derivative. Even more preferred groups are n-halo(preferably n-bromo)-C₄-C₂₀ (preferably C₆-C₁₄) alkanoic acids or esters thereof. The reaction is reacting the amino modified acyl group with the polysaccharide under either etherification or amino alkylation conditions in a substantially water immiscible organic solvent, such as THF substantially 1:1 to 1:10 molecular ratio of polysaccharide chain to bridging group reactant. Preferably, DMF and a base are used to promote an amination reaction at ambient to mild reaction temperatures (less than 100 degrees C.). The reaction may proceed at the interface between the two immiscible solutions, or in solution, to provide a condensation and produce the polysaccharide derivative or analogue. It has been discovered that this reaction at the interface of the organic solution and the aqueous solution imparts a specificity to the reaction for primary alcohol groups of the polysaccharide. A miscible solvent such as THF, DMF, an ether or the like, favors alkylation of the amino groups to form secondary and/or tertiary amines.

By appropriate selection of the type of bridging group reactant and reaction conditions, different structural groups with various chemical properties can be incorporated into the resulting modifying groups and the features of the fiber can be fine-tuned, as desired. Reaction temperatures and other reactions conditions, as well are reactant proportions are well within the skill of the ordinary polymer chemist practitioner in view of the present description of the invention. Other groups and modifications will be apparent to one of ordinary skill in the art from the above discussion.

The anti-cholesterol activity of the fiber, the oil absorbing and the ability to disperse in an aqueous solution may be determined by well-known in vitro and in vivo assays.

Pharmaceutical Compositions and Edible Compositions

In one aspect, the present invention provides a sports drink, snack, nutrient supplement, food or power which may be formulated to contain a cholesterol lowering and fat absorbing therapeutically effective amount of the fiber composition according to the invention.

In another aspect the present invention relates to pharmaceutical compositions comprising a lipase inhibiting effective amount of at least one lipase inhibitor which is added alone or coupled to a digestively non-absorbable moiety. Preferred are such pharmaceutical compositions, comprising an effective amount of a lipase inhibitor with a bio-friendly, bio-compatible, pharmaceutically acceptable fiber moiety, such as a modified polysaccharide comprising acyl groups, wherein the lipase is essentially non-absorbable by the digestive system of an animal such as a dog, cat, non-human primate or humans. The pharmaceutical composition can be administrated to a patent prior to or within one hour of consuming a fat-containing meal to prevent absorption of up to more than one-third of the dietary fat consumed at the meal, and to lower the serum cholesterol with regular use.

In still another aspect, the present invention relates to a method for treating adiposity or obesity by administering to a patient before a fat-contain meal, or up to one hour after such a meal is consumed the present compositions.

Particularly preferred for modifications as polysaccharide fibers are at least one member selected from the group consisting of dextrans, molecular microcrystalline cellulose, wheat bran, oat bran, defatted rice germ, alginic acid, pectin, amylopectin, chitin, crude cellulose, argar, chitosan and the like. Particularly preferred are non-absorbable poly-D-glucosamine or modified poly-D-glucosamine fibers having a derivatized nitrogen, acid or alcohol group and containing at least one acyl group for 1%-15% of the amino groups present in the fiber. Preferred bound polymer moities are derivatized to have an excess of acyl organic acid side chains which are adequate to cause the compound to absorb both oil and water, and tend to disperse evenly in a digestive environment, rather than forming a blob or gel.

The compounds of this invention may be isolated as the free acid or base or converted to salts of various inorganic and organic acids and bases. Such salts are within the scope of this invention. Non-toxic and physiologically compatible salts are particularly useful although other less desirable salts may have use in the processes of isolation and purification.

Numerous methods are useful for the preparation of the salts described above and are known to those skilled in the art. For example, free acid or free base forms of a compound of one of the above compounds can be reacted with one or more molar equivalents of the desired acid or base in a solvent or solvent mixture in which the salt is insoluble, or in a solvent like water after which the solvent is removed by evaporation, distillation or freeze drying.

Alternatively, the free acid or base form of the product may be passed over an ion exchange resin to form the desired salt or one salt form of the product may be converted to another using the same general process.

Prodrug Derivatives of Compounds

This invention also encompasses prodrug derivatives of the therapeutic compounds contained herein. The term “prodrug” refers to a pharmacologically inactive derivative of a parent drug molecule that requires biotransformation, either spontaneous, acid/base reaction, or enzymatic, within the organism to release the active drug. Prodrugs are variations or derivatives of the compounds of this invention which have groups cleavable under digestive system conditions. Prodrugs become the compounds of the invention which are pharmaceutically active in vivo, when they undergo solvolysis under physiological conditions or undergo enzymatic degradation. Prodrug compounds of this invention may be called single, double, triple etc., depending on the number of biotransformation steps required to release the active drug within the organism, and indicating the number of functionalities present in a precursor-type form. Prodrug forms often offer advantages of solubility, digestive compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability.

Formulations of the compounds of this invention are prepared for storage or administration by mixing the compound having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween, Pluronics or polyethyleneglycol.

Dosage formulations of the compounds of this invention to be used for therapeutic administration must be sterile. Sterility may be readily accomplished by filtration through sterile membranes such as 0.2 micron membranes, or by other conventional methods. Fibers may be purified by antiseptic solutions and the fibers may be compounded to provide a powder or granular appearance that is acceptable for formulations. The pH of the preparations of this invention typically will be 3-11, more preferably 5-9 and most preferably 7-8

Therapeutically effective dosages may be determined by either in vitro or in vivo methods. For each particular compound of the present invention, individual determinations may be made to determine the optimal dosage required. The range of therapeutically effective dosages will be influenced by the route of administration, the therapeutic objectives and the condition of the patient. Accordingly, it may be necessary for the therapist to titer the dosage and modify the means of administration as required to obtain the optimal therapeutic effect. The determination of effective dosage levels, that is, the dosage levels necessary to achieve the desired result, will be readily determined by one skilled in the art. Typically, applications of compound are commenced at lower dosage levels, with dosage levels being increased until the desired effect is achieved.

The compounds of the invention can be administered orally in an effective amount within the dosage range of about 10 to 400 mg/kg, preferably about 20 to 200 mg/kg and more preferably about 20 to 50 mg/kg per fat containing meal on a regimen in a single or 2 to 4 divided daily doses. A preferred dosage is an amount (e.g. about 20 to 40 mg/kg) in combination with a lipase having a similar lipase inhibiting effect to the lipase inhibition of 120 mg (approximately 1-2 mg/kg dosage) of orally taken Orlistat. The determination of such equivalent lipase inhibition can be determined via well-known lipase inhibition assays, and may be either an in vivo assay, an in vitro assay, or both. The superior dietary fat calorie reduction and fat absorption properties of the fiber according to the invention can be observed by comparing the amount of anal oil discharged in a patient taking a lipase inhibitor and the fiber according to the invention as compared to an equivalent weight amount Chitosan fiber in a patient taking only Orlistat. The grooming of mice with anal oil is one comparison as compared to Orlistat or the actual comparison of anal discharge in animals or patients also will show a reduction in the amount of oily anal discharge when a fiber according to the invention is administered.

Serum cholesterol can be measured in a number of ways, as well as tissue cholesterol level, or with acceptable equivalent in vitro tests. The ability of the present fiber to lower the serum cholesterol in mammals who regularly consumer the fiber is readily demonstrated.

Typically, for a unit dose form, about 500 mg to 3 g of a compound or mixture of compounds of this invention, as the free acid or base form or as a pharmaceutically acceptable salt, is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

Typical adjuvants which may be incorporated into tablets, capsules and the like are binders such as acacia, corn starch or gelatin, and excipients such as microcrystalline cellulose, disintegrating agents like corn starch or alginic acid, lubricants such as magnesium stearate, sweetening agents such as sucrose or lactose, or flavoring agents. When a dosage form is a capsule, in addition to the above materials it may also contain liquid carriers such as water, saline, oil with fat soluble vitamins, or the like. Other materials of various types may be used as coatings or as modifiers of the physical form of the dosage unit. Sterile compositions for injection can be formulated according to conventional pharmaceutical practice. Buffers, preservatives, antioxidants and the like can be incorporated according to accepted pharmaceutical practice.

In certain aspects of this invention, compounds are provided which are useful as diagnostic reagents to determine lipase activity. In another aspect, the present invention includes pharmaceutical compositions comprising a pharmaceutically effective amount of the compounds of this invention and a pharmaceutically acceptable carrier. In yet another aspect, the present invention includes methods comprising using the above compounds and pharmaceutical compositions for preventing or treating disease states characterized by higher than desired cholesterol levels, or characterized by undesired lipid or fat absorption such as obesity, hyperlipaemia, atherosclerosis and ateioscherosis disorders of the blood coagulation process in mammals, or for stabilizing fats by preventing lipase function in stored fat products and samples. Optionally, the methods of this invention comprise administering the pharmaceutical composition in combination with an additional therapeutic agent such as a traditional anti-cholesterol agent, appetite suppressant, metabolic stimulant and the like.

The preferred compounds also include their pharmaceutically acceptable isomers, hydrates, solvates, salts and prodrug derivatives.

In one embodiment the present invention provides a pharmaceutical composition comprising at least one pharmaceutically acceptable carrier excipient and an amount of at least one of the above described compounds according to the invention in a therapeutically effective amount with respect to limiting or preventing the absorption of some dietary fat. In a preferred embodiment, the pharmaceutical composition comprises a therapeutically effective amount of slow-release lipoprotein lipase, preferably from a microbial or plant source, which selectively hydrolyzes terminal triglyceride groups in combination with an oil absorbing effective amount of polysaccharide such as chitosan, wherein the lipoprotein lipase is present in a ratio of less that 25% with respect to the oil absorbing polysaccharide.

In another embodiment the present invention provides a pharmaceutical composition comprising at least one pharmaceutically acceptable carrier excipient, an amount of at least one of the above described compounds according to the invention in a therapeutically effective amount with respect to lower cholesterol and in an amount capable of limiting or preventing the absorption of some dietary fat, and an oil absorbing effective amount of polysaccharide such as chitosan, wherein such lipase inhibitor is selectively effective to inhibit lipases other than lipases involved in the hydrolysis of terminal triglyceride groups and such lipase inhibitor does not substantially inhibit the absorption of vitamins A, D and E.

In another embodiment the present invention provides a method of using such compounds and pharmaceutical compositions as therapeutic agents for disease states in a mammal having at least one disorder that is due to undesired absorption of dietary fat or for reducing the effective caloric intake of a mammal who consumes dietary fat, which method may be useful in the treatment of undesired weight gain or obesity.

The pharmaceutical compositions comprising a therapeutic amount of the fiber according to the invention may also be used as intermediates in the formation of compounds that may be administered as useful food additives. Such pharmaceutical compositions can be utilized in vivo, ordinarily in mammals such as primates, (non-human and humans), sheep, horses, cattle, pigs, dogs, cats, rats and mice, or in vitro.

The starting materials used in above processes are commercially available from chemical vendors such as Aldrich, Sigma, Lancaster, TCI, and the like, or may be readily synthesized by known procedures, for example, by using procedures such as indicated above.

Reactions are carried out in standard laboratory glassware and reaction vessels under reaction conditions of standard temperature and pressure, except where otherwise indicated, or is well-known in literature available in the art. Further, the above procedures of the claimed invention processes my be carried out on a commercial scale by utilizing reactors and standard scale-up equipment available in the art for producing large amounts of compounds in the commercial environment. Such equipment and scale-up procedures are well-known to the ordinary practitioner in the field of commercial chemical production.

Amino coupling reactions are well-known in the art. Moreover, specific steps that are set forth in the preferred embodiment reaction scheme described above. The reaction products are isolated and purified by conventional methods, typically by solvent extraction into a compatible solvent. Preferred solvents are lower alkane ethers and alcohols; ethyl ether and isopropyl alcohol are preferred for solvent extraction or recrystallization procedures. Esters of carboxylic acid side groups may be formed that permit selective separation of the R and S enantiomers by solvent extraction or recrystallization.

Compositions and Formulations

The compounds of this invention may be isolated as the free acid or base or converted to salts of various inorganic and organic acids and bases. Such salts are within the scope of this invention. Non-toxic and physiologically compatible salts are particularly useful although other less desirable salts may have use in the processes of isolation and purification.

A number of methods are useful for the preparation of the salts described above and are known to those skilled in the art. For example, reaction of the free acid or free base form of a compound of the structures recited above with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product may be passed over an ion exchange resin to form the desired salt or one salt form of the product may be converted to another using the same general process.

Diagnostic applications of the compounds of this invention will typically utilize formulations such as solution or suspension. In the management of undesired fat absorption the compounds of this invention may be utilized in compositions such as tablets, capsules or elixirs for oral administration, sterile solutions or suspensions, and the like, or incorporated into shaped articles. Subjects in need of treatment (typically mammalian) using the compounds of this invention can be administered dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Formulations of the compounds of this invention are prepared for storage or administration by mixing the compound having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween, Pluronics or polyethyleneglycol.

Dosage formulations of the compounds of this invention to be used for therapeutic administration must be sterile. Sterility is readily accomplished by filtration through sterile membranes such as 0.2 micron membranes, by washing with an antiseptic, or by other conventional methods. Formulations typically will be stored in a dry or lyophilized form or in a solution. The pH of the preparations of this invention typically will be between 3 and 11, more preferably from 5 to 9 and most preferably from 7 to 8. While the preferred route of administration is by oral tablets, capsules or other unit dose mechanisms, such as liquids, other methods of administration are also anticipated such as in food stuffs, employing a variety of dosage forms. The compounds of this invention are desirably incorporated into food articles which may include fats for flavoring, but prevent their absorption.

The compounds of this invention may also be coupled with suitable polymers to enhance their therapeutic effects. Such polymers can include lipophilic polymers, such as polysaccharides and the like.

Therapeutically effective dosages may be determined by either in vitro or in vivo methods. For each particular compound of the present invention, individual determinations may be made to determine the optimal dosage required. The range of therapeutically effective dosages will naturally be influenced by the route of administration, the therapeutic objectives, and the condition of the patient. For routes of administration, the cholesterol lower activity, the lipase inhibitor activity, and the fat/oil absorbing ability, in view of the amount of fat consumed, must be individually determined for each inhibitor by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be within the ambit of one skilled in the art. Typically, applications of compound are commenced at lower dosage levels, with dosage levels being increased until the desired effect is achieved.

Typically, about 500 mg to 3 g of a cholesterol lowering fiber compound or mixture of one or more a lipase inhibitor compounds in combination with the fiber of this invention, as the free acid or base form or as a pharmaceutically acceptable salt, is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained. The addition, one or more other therapeutic ingredients such as a fat absorbing polysaccharide or fiber, a fat-specific lipase inhibitor or lipase, as well as other dietary agents may be utilized in therapeutically effective amounts.

Typical adjuvants which may be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. When a dosage form is a capsule, in addition to the above materials it may also contain a liquid carrier such as water, saline, a fatty oil. Other materials of various types may be used as coatings or as modifiers of the physical form of the dosage unit. Sterile compositions for injection can be formulated according to conventional pharmaceutical practice. Buffers, preservatives, antioxidants and the like can be incorporated according to accepted pharmaceutical practice.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice, such as

The compounds of this invention can be utilized in vivo, ordinarily in mammals such as non-human primates, humans, sheep, horses, cattle, pigs, dogs, cats, rats and mice, or in vitro.

The following non-limiting examples are provided to better illustrate the present invention.

### EXAMPLE 1

To a 1 liter flask was added 20 g of chitosan that had been dissolved in 350 mL of DMF (N,N-dimethylformamide), with stirring and the temperature was raised to 50° C. A mixture of 0.3 g of NaOH and 1.5 g of 6-bromohexanoic acid in 20 mL of DMF was added slowly over 30 minutes with stirring. The reaction mixture was stirred at 50° C. for 4 hours. The reaction mixture was cooled to room temperature and poured into 500 mL of ethanol. The solid is suction filtered and washed three times with cold ethanol. The crude precipitate was treated in 1N NaOH ethanol solution for 3 hours, then the pH was reduced to neutral by the addition of 1N HCl. The solid was washed with cold ethanol and H₂O (4:1 ratio) 3 times and dried to provide 19.8 g of functionalized chitosan.

### EXAMPLE 2

To a 1 liter flask was added 20 g of chitosan that had been dissolved in 375 mL of DMF (N,N-dimethylformamide), with stirring and the temperature is raised to 50° C. A mixture of 0.6 g of NaOH and 3 g of 6-bromohexanoic acid in 30 mL of DMF is added slowly over 30 minutes with stirring. The reaction mixture is stirred at 50° C. for 4 hours. The reaction mixture is cooled to room temperature and poured into 500 mL of ethanol. The solid is suction filtered and washed three times with cold ethanol. The crude precipitate is treated in 1N NaOH ethanol solution for 3 hours, then the pH is reduced to neutral by the addition of 1N HCl. The solid is washed with cold ethanol and H₂O (4:1 ratio) 3 times and dried to provide 20.4 g of functionalized chitosan.

### EXAMPLE 3

To a 1 liter flask was added 20 g of chitosan that had been dissolved in 375 mL of DMF (N,N-dimethylformamide), with stirring and the temperature is raised to 50° C. A mixture of 0.4 g of NaOH and 3 g of 8-chlorooctanoic acid in 30 mL of DMF is added slowly over 30 minutes with stirring. The reaction mixture is stirred at 50° C. for 5 hours. The reaction mixture is cooled to room temperature and poured into 500 mL of ethanol. The solid is suction filtered and washed three times with cold ethanol. The crude precipitate is treated in 1N NaOH ethanol solution for 3 hours, then the pH is reduced to neutral by the addition of 1N HCl. The solid is washed with cold ethanol and H₂O (4:1 ratio) 3 times and dried to provide 21.3 g of functionalized chitosan.

### EXAMPLE 4

To a 1 liter flask was added 20 g of chitosan that had been dissolved in 375 mL of DMF (N,N-dimethylformamide), with stirring and the temperature is raised to 50° C. A mixture of 0.6 g of NaOH and 3 g of 6-bromohexanoic acid in 30 mL of DMF is added slowly over 30 minutes with stirring. The reaction mixture is stirred at 50° C. for 4 hours. The reaction mixture is cooled to room temperature and poured into 500 mL of ethanol. The solid is suction filtered and washed three times with cold ethanol. The crude precipitate is treated in 1N NaOH ethanol solution for 3 hours, then the pH is reduced to neutral by the addition of 1N HCl. The solid is washed with cold ethanol and H₂O (4:1 ratio) 3 times and dried to provide 20.4 g of functionalized chitosan.

### IN VITRO, BIOLOGICAL AND OTHER PROPERTIES ASSAY EXAMPLES

### EXAMPLE 5

### IN VITRO EXAMPLE

The N-hexanoic acid modified Chitosan (N-HMC) of Example 1 was compared to its ordinary Chitosan starting material with respect to dissolving in oil and aqueous dispersion. The N-HMC (500 mg) easily dissolved or dispersed in 30 g of olive oil after only a few mild with a glass stirring rod to form a cloudy uniform mixture. By contrast, extreme agitation was required including vigorous shaking to provide dissolving or dispersion of the ordinary Chitosan starting material, and more than half of the oil floated to the top of the container, which indicates that this was too much oil for ordinary Chitosan to absorb.

When 20 ml of distilled water was added to the N-HMC/olive oil mixture with mild stirring a uniform solution of water, oil and N-HMC is obtained. Adding water soluble coloring shows even distribution of the water throughout the oil mixture, instead of floating to the bottom as is usually with water and oil mixtures.

When 20 ml of distilled water was added to the ordinary Chitosan/olive oil mixture, it was not possible to stir the water and oil layers together and the oil quickly rises to the top, while water goes to the bottom, leaving an oil/Chitosan mixture as a middle layer sandwiched between the oil and water layers.

### IN VIVO EXAMPLES

### EXAMPLE 6

The N-hexanoic acid modified Chitosan (N-HMC) of Example 1 is administered to mice by gavage along with radioactive oil to determine its ability to absorbing radioactive oil in the digestive systems of mice and eliminate the dietary oil in the waste without the oil being metabolic fat calories. The radioactivity of the digestive waste is measured for mice given the N-HMC and compared with that of normal mice to determine the amount of oil that is being removed in the digestive waste by N-HMC. The data shows that N-HMC removes more than 30 times its weight in digestive oil compared to normal mice that were not administered any N-HMC. Since Chitosan is reported in the literature as being capable of removing up to 8 times it weight in dietary oil, the N-HMC is significantly more potent. In addition, the waste pellets of the mice were normal and perhaps a little more soft than those of the control mice. This indicates that the N-HMC is also a dietary fiber that may be a mild stool softener as compared to other fibers that only provide bulk without a softening effect.

### EXAMPLE 7

The N-hexanoic acid modified Chitosan (N-HMC) dietary supplement of Example 1 is taken by healthy volunteers who do not ordinarily consume large amounts of simple carbohydrates (since one large soft drink can contain one-half of the average daily caloric metabolic requirements) and ordinarily have a moderate to high fat diet. The amount of the N-HMC taken is capable of binding 50% or more of the average amount of dietary fat ordinarily consumed in their diet. Without other changes to dietary habits (other than prudent moderation of simple carbohydrates), the amount of weight lost by volunteers is profound. The N-HMC dietary fiber supplement is observed to be dose dependant and subjects average about one-half to two pounds of weight loss per week, depending upon the amount of N-HMC fiber consumed and the amount of dietary fat that is ordinarily consumed in their diet. No side effects are observed with any of the volunteers other. A positive report of mild stool softening and promotion of bowel elimination regularity is reported.

### EXAMPLE 8

Cholesterol binding and in vivo serum cholesterol lowering assays are performed using standard methods in the art. Lipase inhibition assays were performed utilizing lipase inhibition kits that are available from Aldrich or Sigma. Oil binding is demonstrated with olive oil, water and water soluble dyes.

In view of the above description it is believed that one of ordinary skill can practice the invention. The examples given above are non-limiting in that one of ordinary skill in view of the above will readily envision other permutations and variations on the invention without departing from the principal concepts. Such permutations and variations are also within the scope of the present invention.

What is claimed is:

1. A pharmaceutical composition comprising a pharmaceutically acceptable carrier and an effective amount of an N-Acyl modified poly-D-glucosamine polysaccharide dietary fiber that can absorb more than 10 times is weight in dietary fat or oil and remove dietary fat in a dose dependent manner without permitting the fat to be metabolized as caloric dietary fat.

2. A composition according to claim 1, wherein the polysaccharide fiber is a poly-D-glucosamine or derivative thereof and at least one hydrogen atom on from 3% to 10% on the amine groups (—NH2 groups) of the repeating D-glucosamine or modified D-glucosamine groups have been replaced by a 4-20 carbon atom alkyl group comprising at least one carboxylic group, wherein the carboxylic group may be esterfied by a lower alcohol group or may be in a salt form, in order to form N-alkylacyl groups, or modified N-alkylacyl groups, on the polymer backbone.

3. A composition according to claim 2, wherein the N-alkylacyl group on the modified poly-D-glucosamine backbone is a member selected from the group consisting of an N-6-hexanoic acid group, an N-8-octanoic acid group, an N-11-undecanoic group, or a combination thereof, wherein the acid group many be the free acid, an ester, or a salt thereof.

4. A composition according to claim 2, wherein the N-alkylacyl groups on the modified poly-D-glucosamine backbone comprise an N-6-hexanoic acid group, wherein the acid group many be the free acid, an ester, or a salt thereof, and from 5% to 10% of the amino groups on the poly-D-glucosamine backbone are modified to form N-alkylacyl groups in the form of a free acid, an ester or a salt thereof.

5. A composition according to claim 4, wherein the modified poly-D-glucosamine fiber is capable of dispersing in an aqueous environment with mild agitation and is capable of eliminating more than 20 times its weight in dietary fat in the dietary waste without the dietary fat being metabolized as caloric dietary fat.

6. A method for lowering the serum cholesterol in a patient by treating the patient with an effective amount of the composition according to claim 1.

7. A method of increasing the digestive health of a mammal by treating said mammal with an amount of the composition of claim 1 that is effective as a dietary fiber or supplement.

8. A method according to claim 6, wherein the composition is administered to the mammal in a dosage from about 250 milligrams to 3 grams per meal.

9. A method according to claim 8, wherein the composition is administered in a dosage from about 500 milligrams to 2 grams per meal.

10. A method according to claim 9, wherein the composition is administered in a dosage from about 750 mg to 1 g per meal.

11. A composition according to claim 1, further comprising an amount of a lipase inhibitor effective for the treatment of obesity in admixture therewith.

12. A bulk chemical composition comprising an N-Acyl modified poly-D-glucosamine polysaccharide dietary fiber as described in claim 1, alone, or in combination with a pharmaceutically acceptable carrier or excipient.
